(19) United States
(12) Patent Application Publication
(10) Pub. No.: US 2005/0209441 A1
(43) Pub. Date: Sep. 22, 2005

(54) Process for promoting proper folding of human serum albumin using a human serum albumin ligand

(76) Inventor: Jackson D. Lile, Boulder, CO (US)

Correspondence Address: HOLLAND & HART, LLP, 555 17TH STREET, SUITE 3200, DENVER, CO, 80201, US

(21) Appl. No.: 11/085,291

(22) Filed: Mar. 21, 2005

Related U.S. Application Data
(60) Provisional application No. 60/555,450, filed on Mar. 22, 2004.

(51) Int. Cl. C07K 14/765

(52) U.S. Cl. 530/363

(57) ABSTRACT

The present invention is a process for refolding and renaturing human serum albumin protein to substantially native conformation by the addition of a human serum albumin refolding ligand to a solution containing the protein under conditions conducive to refolding of the protein.

### CROSS REFERENCE TO RELATED APPLICATION

This application claims the benefit of the filing date of U.S. Provisional Patent Application Ser. No. 60/555,450, filed Mar. 22, 2004.

### TECHNICAL FIELD OF THE INVENTION

The present invention relates to methods for manufacturing recombinant serum albumin proteins, and particularly to the field of refolding recombinant human serum albumin proteins.

### BACKGROUND OF THE INVENTION

Human serum albumin (hereinafter referred to simply as HSA) is the most abundant protein contained in plasma. It is produced in the liver, and contributes to the maintenance of osmotic pressure in blood and binds to nutrients and metabolites to thereby transport these substances. HSA has been utilized therapeutically in the treatment of such indications as hypoalbuminemia (caused by an albumin loss or reduction in albumin synthesis) and hemorrhagic shock.

Currently, HSA is produced primarily as a fractionated product of collected blood, which is uneconomical and is subject to a sporadic supply of blood. In addition, collected blood sometimes contains undesirable pathogenic substances, such as hepatitis or HIV virus. Recent advances in recombinant DNA techniques, however, have made possible the production of various types of useful polypeptides by microbial host cells. Establishing techniques for the large scale production of HSA by recombinant methods and subsequent high grade purification would therefore be desirable.

Recombinant mammalian proteins can be expressed in either prokaryotic host systems, such as E. coli, or eukaryotic host systems, such as yeast or mammalian cells. Eukaryotic systems possess the cellular machinery to ensure proper folding and most post-translational modifications, but typically result in low yields and require complex and expensive purification procedures. In contrast, expression in prokaryotic systems results in much higher yields, but the cellular machinery necessary for proper folding of eukaryotic proteins is lacking.

Some recombinant mammalian proteins will fold spontaneously to the native conformation, and can therefore be expressed in prokaryotic systems with little difficulty. Other proteins, such as large proteins and proteins with multiple intra-molecular disulfide bonds (covalent bonds between two cysteine amino acid residues at different locations within the protein), however, are more problematic. Such proteins, when formed in a prokaryote cell, form large insoluble inclusion bodies (constituting up to 80% of the net weight of the cell) comprised of an aggregation of unfolded or incorrectly folded proteins bound together by the formation of incorrect intra- or inter-molecular disulfide bonds. Such proteins, of course, are not biologically active. Although inclusion bodies can be readily purified by centrifugation on the basis of their relatively high density, the proteins forming the inclusion body are not folded in the native conformation and must therefore be refolded into its biologically active native conformation.

A variety of methods have been used to re-solubilize and refold proteins to a biologically active native conformation. Typically, the refolding process begins with washed inclusion bodies, which are solubilized with high concentrations of denaturants such as mercaptoethanol, guanidine hydrochloride or urea. The amount of aggregation may continue to increase with time if the protein is allowed to remain in the denaturant, and incorrect disulfide bonds may slow the refolding process or possibly generate kinetically trapped intermediates that are difficult to reverse. Removal of the denaturant from the solubilized inclusion bodies by dialysis or desalting columns will cause the protein to precipitate under conditions where the native protein needs to be refolded. A misfolded protein solution can also have a very low specific activity in biological assays.

HSA is a particularly difficult protein to refold, primarily because it has 17 disulfide bonds (35 cysteine residues in total) that can incorrectly form in various combinations. Lee et al. (J. Bio. Chem. 267:14753-14758, 1992) disclose a process for refolding denatured/disulfide-reduced HSA by first completely reducing the protein with glutathione, and then completely oxidizing the protein with oxidized glutathione, over a long period of about 48 hours.

U.S. Pat. No. 6,617,133 (Noda et al.) also disclose a process for purifying recombinant human serum albumin by heating a culture medium containing rHSA and the rHSA-producing host cells, feeding the heated solution upwardly into a fluidized bed in which adsorbent particles are suspended to effect contacting with the adsorbent particles, and then recovering the adsorbed fraction containing the rHSA.

Both of the above processes require lengthy periods of time to achieve proper refolding, which is unacceptable for commercial production of rHSA. Accordingly, a rapid method of producing recombinant human serum albumin is desired.

### SUMMARY OF THE INVENTION

The present invention is a process for refolding HSA to substantially native conformation using a human serum albumin ligand. More particularly, the present invention is a process for refolding and renaturing human serum albumin protein to substantially native conformation by the addition of a human serum albumin refolding ligand to a solution containing the protein under conditions conducive to refolding of the protein.

In a particular embodiment of the present invention, a human serum albumin refolding ligand is added in the course of a process involving the following three stages: (a) solubilizing human serum albumin protein in a solution comprising a denaturant and a first thiol reducing compound at concentrations sufficient to disrupt formation of all disulfide bonds and form free thiols, (b) decreasing the concentration of the denaturant, at pH greater than about 9.5, adding to the solution a disulfide oxidizing compound to a molar concentration sufficient to create mild oxidizing redox conditions to oxidize a portion of the free thiols and form mixed native and non-native disulfide bonds, and adding a human serum albumin refolding ligand at a time from stage (b) to (c); and (c) further decreasing the concentration of the denaturant, lowering the pH of the solution to less than 9.5, and adding a second thiol reducing compound to a molar concentration sufficient to create mild reducing conditions to catalyze interchange of the non-native disulfide bonds and form native disulfide bonds, thereby providing a human serum albumin protein having substantially native conformation.

### DETAILED DESCRIPTION OF THE INVENTION

Recombinant Expression of HSA

The process of the present invention is used to assist in refolding human serum albumin (HSA) protein that is incompletely or incorrectly folded, for example, HSA that has been purified from natural sources or, more commonly, HSA that has been produced by recombinant expression in host cells. The use of recombinant host cells to produce proteins is a highly cost effective method for obtaining large quantities of a protein for use in commercial applications. Recombinant HSA proteins are typically expressed in a suitable host, for example, eukaryotic cells (such as yeast and animal cells), or procaryotic cells (such as E. coli or other type of bacteria), using a standard expression vector such as a plasmid, bacteriophage or naked DNA, and the protein expressed from the plasmid or DNA integrated into the host chromosome. Expression in prokaryotic systems typically provides higher yields of the protein at lower cost, but suffers from the disadvantage that prokaryotic systems lack the cellular machinery to correctly fold complex mammalian proteins. Some eukaryotic host systems may also not result in correctly folded proteins. The process of the present invention is therefore applicable to any method of producing HSA that results in an HSA protein that is not folded in the native conformation, but is particularly advantageous in promoting folding of HSA proteins expressed in prokaryotic systems.

Suitable methods for recombinant expression of HSA are well known in the art. Bacterial strains for expression of HSA are commercially available or can be obtained from the American Type Culture Collection (ATCC), 10801 University Boulevard, Manassas, Va. 20110-2209. Expression of recombinant proteins in host cells also requires suitable expression vectors, which can be obtained from any number of sources, including the ATCC. Expression vectors require a promoter to insure that the DNA is expressed in the host, and may also include other regulatory sequences that affect the expression of the recombinant protein. The vector may also include means for detection, such as an antibiotic resistance marker, green fluorescent protein tag, or antigen tag to facilitate in purification of the recombinant protein. Once the DNA encoding the protein to be purified is introduced into the host, the host is cultured under appropriate conditions until sufficient amounts of recombinant protein are obtained.

Preparation of HSA-producing host, production of rHSA by its culturing and isolation and recovery of rHSA from the cultured broth are all carried out in accordance with known methods which may be modified for optimization. For example, preparation of an HSA-producing host may be effected using a process in which a natural HSA gene is used (EP-A-73646, EP-A-79739 and EP-A-91527), a process in which a modified human serum albumin gene is used (EP-A-206733), a process in which a synthetic signal sequence is used (EP-A-329127), a process in which a serum albumin signal sequence is used (EP-A-319641), a process in which a recombinant plasmid is introduced into a chromosome (EP-A-399455), a process in which hosts are fused (EP-A-409156), a process in which a mutation is generated in a methanol containing medium, a process in which a mutant AOX₂ promoter is used (EP-A-506040), and a process in which HSA is expressed in B. subtilis (EP-A-229712).

Culturing of the HSA-producing host may be effected by each of the methods disclosed in the above patents, by a method in which producer cells and the product are obtained in high concentrations by a fed-batch culture (a semi-batch culture) which method is carried out by gradually supplying a high concentration solution of glucose, methanol or the like in appropriate small amounts to avoid high concentration substrate inhibition against the producer cells (JP-A-3-83595) or by a method in which the HSA productivity is improved by the addition of fatty acids to the culture medium (EP-A-504823 and U.S. Pat. No. 5,334,512).

A medium usually employed in the art supplemented with a fatty acid having from 10 to 26 carbon atoms or a salt thereof can be used as a medium for culturing a transformed host, and culturing of the transformant can be carried out under known conditions. The medium may be either synthetic or natural, but preferably is a liquid medium. For example, a suitable synthetic medium may be composed of: carbon sources, such as various saccharides; nitrogen sources, such as urea, ammonium salts, nitrates; trace nutrients, such as various vitamins, nucleotides; and inorganic salts, such as of Mg, Ca, Fe, Na, K, Mn, Co and Cu. An illustrative example of such a medium is YNB liquid medium, which consists of 0.7% Yeast Nitrogen Base (Difco) and 2% glucose. An illustrative example of a useful natural medium is YPD liquid medium, which consists of 1% Yeast Extract (Difco), 2% Bacto Peptone (Difco) and 2% glucose. The medium pH may be neutral, weakly basic or weakly acidic. In the case of a methylotrophic host, the medium may be further supplemented with methanol in an amount of approximately from 0.01 to 5%.

The culturing temperature preferably ranges from 15 to 43.degree.C. (20 to 30° C. for yeast, 20 to 37° C. for bacterium). The culturing period ranges from 1 to 1,000 hours, preferably 20 to 360 hours, by means of static or shake culturing or batch, semi-batch or continuous culturing under agitation and aeration. It is desirable to prepare a seed culture prior to the batch culturing by means of static or shake culturing or batch, semi-batch or continuous culturing under agitation and aeration. The seed culturing may be carried out using the aforementioned YNB liquid medium or YPD liquid medium, preferably at 30° C. (for yeast) or 37° C. (for bacterium) and for 10 to 100 hours.

U.S. Pat. No. 6,617,133 (Noda et al.) disclose a process for purifying recombinant human serum albumin by heating a-culture medium containing rHSA and the rHSA-producing host cells, feeding said heated solution upwardly into a fluidized bed in which adsorbent particles are suspended to effect contacting with the adsorbent particles and then recovering the adsorbed fraction containing the rHSA, and a composition comprising rHSA which shows a A350/A280 ratio of below 0.015, when formulated into a 25% solution of albumin.

The production of rHSA in microorganisms has been disclosed in EP 330 451 and EP 361 991. Purification techniques for rHSA have been disclosed in: WO 92/04367, removal of matrix-derived dye; EP 464 590, removal of yeast-derived colorants: and EP 319 067, alkaline precipitation and subsequent application of the rHSA to a lipophilic phase having specific affinity for albumin.

Once the protein has been expressed in the maximum amount, it must be separated and purified from the bacterial host. The protein is isolated generally by lysing the cells, for example, by suspending in detergent, adding lysozyme, and then freezing (for example, by suspending cells in 20 ml of TN/1% Triton™ X-100, adding 10 mg lysozyme and freezing at −20° C. overnight), thawing and adding DNAase to degrade all of the bacterial DNA, then washing the resulting precipitate in a buffered solution. The precipitate is then dissolved in an appropriate solution as discussed below, for refolding.

Refolding/Purification Methods

The process of the present invention provides a novel method for refolding HSA to substantially its native conformation. In one embodiment of this process, HSA is subjected to the following processes: (A) HSA is solubilized in a solution comprising sufficient amounts of a denaturant and a first thiol reducing compound to disrupt formation of all disulfide bonds and form free thiols; (B) the concentration of the denaturant is then decreased and a sufficient amount of a disulfide oxidizing compound is added to the solution, at above physiological pH, sufficient to create mild oxidizing redox conditions and thereby oxidize a portion of the free thiols to form mixed native and non-native disulfide bonds; and (C) the concentration of the denaturant is further decreased, the pH of the solution is lowered, and a sufficient amount of a second thiol reducing compound is added to the solution, sufficient to create mild reducing conditions and thereby catalyze interchange of the non-native disulfide bonds to native disulfide bonds. The above process provides a human serum albumin protein having substantially native conformation. In another aspect of the invention, refolding of HSA is further facilitated by the addition of an HSA refolding ligand in step B and or C.

Particular embodiments of the process of the present invention are described below. Particular conditions relating to the embodiments described below can be readily determined by those skilled in the art of protein purification and refolding. In particular, the temperature conditions can be selected as appropriate. While higher temperature conditions may be used to accelerate refolding, temperature conditions significantly greater than room temperature may result in degradation of the protein. In the disclosed embodiments of the present invention, the temperature conditions may be from about 4° C. to about 45° C. In particular embodiments, the temperature conditions are from about 25° C. to about 37° C. In more particular embodiments, the temperature conditions are about 37° C. While particular preferred temperature conditions are disclosed below, such conditions may be adjusted or modified, as appropriate.

Ligand-Assisted Refolding

The present invention relates to a process for refolding HSA protein utilizing one or more HSA binding ligands capable of promoting refolding of HSA. As used herein, the term “HSA refolding ligand” refers to a ligand that binds to and promotes refolding of HSA, particular embodiments of which are disclosed below. An HSA refolding ligand is added at any time from stage B to stage C of the process to accelerate refolding of HSA. In particular embodiments, an HSA refolding ligand is added at stage B, so that the refolding ligand is present in both stage B and the subsequent stage C of the process, thereby optimizing overall yield. In a particular embodiment of the present invention, HSA is refolded to substantially native conformation by providing human serum albumin in a solution under conditions conducive to refolding of human serum albumin, and adding a human serum albumin refolding ligand comprising a human serum albumin site 2 binding ligand. In another embodiment, HSA is refolded to substantially native conformation by providing human serum albumin in a solution comprising sufficient amounts of a denaturant, a first thiol reducing compound, and a disulfide oxidizing compound to create mild oxidizing redox conditions and oxidize a portion of the free thiols to form mixed native and non-native disulfide bonds, and adding a human serum albumin refolding ligand comprising a human serum albumin site 2 binding ligand. Other embodiments of the present invention utilizing an HSA refolding ligand are illustrated below in the context of a staged process for refolding HSA protein.

Suitable HSA refolding ligands may include, for example, the free acids or salts of the n-alkyl C2-C14 mono- and di-fatty acids, the n-alkyl C2-C14 mono- and di-alcohols; ligands that specifically bind HSA site 1, such as warfarin, n-butyl p-aminobenzoate or Indomethacin; ligands that specifically bind HSA site 2, such as Dichlofenac, Ibuprofen, Naproxen, L-thyroxine, L-trytophan, and so forth. Compounds known to bind to other sites are also effective as refolding ligands such as bilirubin, lithocholic acid, lithocholic sulfate, as well as soluble calcium salts. Also, combinations of HSA refolding ligands that bind to different sites of the HSA protein may be used. In particular embodiments of the present invention, the HSA refolding ligand is a ligand that binds to HSA site 2.

In one embodiment of the present invention, HSA protein is refolded using ligands capable of specifically binding HSA site 1, such as 8-anilino-1-naphthalenesulfonic acid, Indomethacin, n-butyl p-aminobenzoate, Warfarin, sodium salicylate, Tolbutamide, sodium valproate, lodipamide, dansyl-L-asparagine, Sulfisoxazole, Phenol Red, Phenylbutazone, and other known site 1 binding ligands. In order to prevent contamination of the purified HSA protein with the above ligands, use of the above ligands to promote refolding is preferably accomplished by immobilizing the ligands on a resin, exposing HSA to the immobilized ligand to promote refolding, and then eluting HSA from the immobilized resin in a purified form.

More particularly, suitable HSA refolding ligands include ligands that bind to HSA site 2. Particular ligands that are known to bind to HSA site 2 include the various fatty alcohols, fatty acids, and salts of fatty acids. The fatty acid salts may be, for example, alkaline earth salts, including sodium salts and potassium salts, as well as other salts, such as ammonium salts. In particular embodiments, the fatty acids salts are sodium salts.

In another embodiment of the present invention, HSA protein is refolded using ligands that bind to site 2 of HSA, such as Ibuprofen, Naproxen, Dichlofenac, L-tryptophan, sodium hippurate, L-thyroxine, indole-3-acetate. Other site 2 binding ligands may also be used. In one embodiment, the site 2 ligand is Ibuprofen, Naproxen, Dichlofenac, L-tryptophan. In yet another embodiment, the site 2 ligand is L-tryptophan, a natural and essential amino acid that might not result in unacceptable contamination of the purified HSA protein. Again, in order to prevent contamination of the purified HSA protein with the above ligands, use of the above ligands to promote refolding is preferably accomplished by immobilizing the ligands on a resin, exposing HSA to the immobilized ligand to promote refolding, and then eluting HSA from the immobilized resin in a purified form.

In another embodiment of the present invention, HSA protein is refolded using n-alkyl fatty acids or their alkaline earth salts capable of binding to HSA. In particular embodiments, the refolding ligand is an n-alkyl fatty acids or their alkaline earth salts capable of binding to site 2 of HSA. Such ligands include sodium myristate, sodium laurate, sodium caprate, sodium caprylate, sodium caproate, sodium butyrate, and sodium acetate. In a particular embodiment of the present invention, the ligand is a C₈-C₁₂ fatty acid, such as sodium laurate, sodium caprate and sodium caprylate. Examples of particular fatty acids are shown below in Table 1.

In the context of protein refolding, acceptable fatty acid salts will generally promote refolding at concentrations equal to or greater than about 0.1 mM. In particular embodiments of the invention, the concentration of fatty acid salts used to promote refolding is greater than about 1 mM, and more preferably greater than about 10 mM. As shown below in Table 1, the acceptable ranges and optimal concentrations will vary according to the choice of refolding ligand. In a particular embodiment, sodium caprate is used as the HSA refolding ligand, at higher concentrations of from about 1 to about 10 mM.

In a particular embodiment of the present invention, HSA protein is refolded using n-alkyl alcohols such as tetradecanol (C14), dodecanol (C12), decanol (C10), octanol (C8), hexanol (C6), pentanol (C5), butanol (C4), propanol (C3), and ethanol (C2). In particular embodiments, C6-C14 alcohols are used to assist refolding. C2-C4 alcohols are more effective at high concentrations, which may be of use for large scale manufacturing. The alcohols may be primary, secondary, or tertiary alcohols (with the OH group bonded to the primary, secondary, or tertiary carbon atom). In other particular embodiments, the n-alkyl alcohols are primary straight chain alcohols, with the OH group bonded to the primary carbon. Such alcohols include, for example, propanol, butanol, and pentanol. Examples of particular alcohols and particular fatty acids, and preferred and optimal concentrations are listed below in Table 1.

TABLE 1

| Refolding Ligand | *ED50 | Preferred Range | Optimal Concentration |
|---|---|---|---|
| Fatty acids | | | |
| Na Laurate (C12) | 45 uM | 10 uM-5 mM | 1 mM |
| Na Caprate (C10) | 125 uM | 30 uM-30 mM | 10 mM |
| Na Caprylate (C8) | 2 mM | 500 uM-30 mM | 30 mM |
| Na Caproate (C6) | 17 mM | 3 mM-100 mM | >30 mM |
| Alcohols | | | |
| 1-Dodecanol (C12) | 230 uM | 50 uM-300 uM (saturated) | 300 uM (saturated) |
| 1-Decanol (C10) | 300 uM | 100 uM-10 mM | 3 mM |
| 1-Octanol (C8) | 450 uM | 100 uM-10 mM | 3 mM |
| 1-Hexanol (C6) | 2 mM | 0.5 mM-100 mM | 25 mM |
| 1-Pentanol (C5) | 25 mM | 5 mM-500 mM | 150 mM |
| 1-Butanol (C4) | 125 mM | 25 mM-1 M | 500 mM |
| 1-Propanol (C3) | 800 mM | 100 mM-1.5 M | >1 M |

*ED50 for refolding is that concentration of compound which gives 50% refolding in 30 minutes at 37° C. with HSA at 1 mg/ml.

In another embodiment of the present invention, HSA protein is refolded using ligands capable of binding HSA at sites other than site 1 or 2, such as bilirubin, lithocholic acid, lithocholic sulfate, and other compounds referred to bile salts.

Particular compounds useful in assisting refolding of HSA proteins include those listed in the following Table 2. Table 2 shows the reported binding constants of site 1 ligands and their observed ED50 values for refolding assistance (ED50 for refolding is that concentration of compound which gives 50% refolding in 30 minutes at 37° C. with HSA at 1 mg/ml). Table 2 shows that site 1 ligands with very similar binding constants may have very different refolding ED50's.

TABLE 2
Site 1 Ligands

| | ED50 for | Binding |
|---|---|---|
| | HSA refolding | constant |
| | Very different | Similar binding |
| Site 1 ligands | refolding ED50's | constants |
| Comparison of 4 | | |
| compounds | | |
| n-butyl p-aminobenzoate | 800 uM | 2.8 × 10.5 |
| Warfarin | 3 mM | 3.4 × 10.5 |
| Na Salicylate | 12 mM | 1.9 × 10.5 |
| Na Valproate | 15 mM | 2.8 × 10.5 |
| Comparison of 2 | | |
| compounds | | |
| Indomethacin | 600 uM | 1.4 × 10.6 |
| Sulfisoxazole | >30 mM | 1 × 10.6 |

Table 3 shows some site 2 ligands with very similar binding constants and very similar refolding ED50's.

TABLE 3
Site 2 Ligands

| | Very similar | Very similar |
|---|---|---|
| Comparison of | refolding | binding |
| 3 compounds | ED50's | constants |
| Ibuprofen | 400 uM | 2.7 × 10.6 |
| Naproxen | 300 uM | 3.7 × 10.6 |
| Dichlofenac | 300 uM | 3.3 × 10.6 |
| | | Binding |
| Comparison of | Refolding ED50 | constant |
| 4 fatty acids | progression | progression |
| Na Laurate | 45 uM | 1.1 × 10.7 |
| Na Caprate | 125 uM | 8.3 × 10.6 |
| Na Caprylate | 2 mM | 1.6 × 10.6 |
| Na Caproate | 17 mM | 7 × 10.4 |

Stage A: HSA Reduction

In stage A of the process, HSA is denatured and reduced to disrupt formation of all disulfide bonds and form free thiols. In particular embodiments of the invention, stage A comprises solubilizing HSA protein in a solution comprising a denaturant and a first thiol reducing compound at concentrations sufficient to disrupt formation of all disulfide bonds and form free thiols. In another embodiment, stage A comprises solubilizing human serum albumin protein in a solution comprising a denaturant, and a first thiol reducing compound at a molar concentration greater than the molar concentration of human serum albumin disulfide bonds, sufficient to disrupt formation of all disulfide bonds and form free thiols. In yet another embodiment, stage A comprises solubilizing human serum albumin protein in a solution comprising urea, and a first thiol reducing compound to a molar concentration greater than the molar concentration of human serum albumin disulfide bonds, sufficient to disrupt formation of all disulfide bonds and form free thiols. The denaturant and first thiol reducing compound may be added to the HSA solution in any desired order.

HSA must first be dissolved in an appropriate buffer solution that is compatible with the HSA protein and the particular reagents selected, which can be readily determined by those skilled in the art of protein purification and refolding. As used in the present invention, compatible buffer solutions include those using sodium bicarbonate, sodium borate, ammonium acetate, and so forth. In particular embodiments, sodium bicarbonate and sodium borate are used. In a more particular embodiment of the present invention, HSA is dissolved in a buffer solution of sodium bicarbonate, which may contain from 5 mM to 500 mM sodium biocarbonate. In another embodiment, the sodium bicarbonate buffer solution may contain from 10 mM to 100 mM sodium biocarbonate. In yet anther embodiment, the sodium bicarbonate buffer solution is about 15 mM sodium bicarbonate.

Inclusion bodies that form during recombinant expression of mammalian protein, such as HSA, contain disulfide bonds that must be disrupted in the presence of reducing reagents. Accordingly, the initial buffer solution of the process of the present invention will also contain a reducing agent to disrupt intramolecular and intermolecular disulfide bonds that form within and between HSA molecules. Representative reducing agents include thiol based reducing agents such as dithiothreitol (DTT), dithioerythritol, 2-mercaptoethanol, cysteine, cysteamine, glutathione, ethanethiol, 1-propanethiol, 3-methyl-1-butanethiol, or a non-thiol based compound such as TCEP (Tris[2-carboxyethyl]phosphine). In particular embodiments of the present invention, dithiotheritol and TCEP are used as reducing agents.

The molar concentration of the reducing agent should be at least equal to the molar concentration of protein disulfide bonds. In particular embodiments of the present invention, the molar ratio of the first reducing agent is greater than the protein disulfide concentration. In another embodiment, the molar ratio to the first reducing agent is from about 1-20 times that of the protein disulfide concentration. In another embodiment, the molar ratio of the reducing agent is from about 1-10 times that of the protein disulfide concentration. In yet another embodiment, the molar ratio of the reducing agent is about 1.5 to 3 times that of the protein disulfide concentration.

Finally, the buffer solution used for the process of the present invention will also contain a chaotropic agent to further assist in disrupting inter- and intra-molecular attractive forces within and between HSA molecules, and thereby dissolve the HSA protein into solution. Suitable chaotropic reagents include, for example, urea, guanidine hydrochloride, and thiocyanate. As used in the process of the present invention, the concentration of urea used to denature protein precipitates and aggregates are greater than about 3 M to about 10 M. In other embodiments, the concentration of urea if from about 5 M to about 8 M. In yet another embodiment, the concentration of urea is about 6 M.

The pH of the buffer solution is also a factor in achieving successful refolding of the HSA protein. Generally, the refolding process is initiated at high pH conditions, sufficient to facilitate dissolution of precipitated or aggregated proteins in the presence of a reducing agent and a chaotrope, such as 6-8 M urea. The particular pH of a buffer will generally be selected to be compatible with other steps in the refolding process. In the refolding process of the present invention, HSA is initially dissolved in a buffer solution at a pH sufficient to dissolve the HSA protein in the presence of a reducing agent and a chaotropic agent. In the illustrated embodiments of the present invention, the initial buffer solution has a pH from about pH 3 to about pH 11, depending on the chosen reducing agent. In one embodiment, the effective pH for reduction with DTT, for example, is from about pH 5 to about pH 11. In another embodiment using DTT as the reducing agent, the pH is from about pH 7 to about pH 11. In yet another embodiment using DTT as the reducing agent, the pH is about pH 10.3. In another embodiment, using TCEP as the reducing agent, the pH is from about pH 3 to about pH 10. In another embodiment using TCEP as the reducing agent, the pH is from about pH 3 to about pH 7. In yet another embodiment using TCEP as the reducing agent, the pH is about pH 4.

In one particular embodiment of the present invention, the buffer solution used in step A contains 15 mM sodium bicarbonate, pH 10.3, containing urea to 6 M and greater than molar excess of reducing agent, such as dithiotheritol (DTT) at 3-fold molar excess of protein disulfides, to reduce HSA protein disulfides.

By way of example, sufficient quantity of HSA can be added to a buffer solution of 15 mM sodium bicarbonate, pH 10.3, containing 6 M urea, to result in an HSA concentration of 10 mg/ml (2.43 mM protein disulfides), and sufficient DTT is added to result in a concentration of 7.3 mM DTT. The HSA protein is then allowed to remain in the solution at a temperature and for a period of time sufficient to reduce and denature, for example, a temperature of 37° C. for a time period of 15-30 minutes.

Stage B: Preliminary Disulfide Shuffling

In stage B of the process of the present invention, the concentration of the denaturant is decreased and a sufficient amount of a disulfide oxidizing compound is added to the solution, at high pH, to create mild oxidizing redox conditions and thereby oxidize a portion of the free thiols to form mixed native and non-native disulfide bonds. This stage can be described as a stage in which the conditions permit some free thiols to form disulfide bonds, including both native and non-native disulfide bonds, and there exists an equilibrium or exchange between the free thiols and the disulfides, referred to herein as “disulfide shuffling.” Disulfide shuffling thus entails the conversion of free thiols to disulfides, as well as the reverse—disulfides to free thiols. In more functional terms, such conditions selectively permit the reversal of the formation of non-native disulfides, which are kinetically disfavored, back to free thiols, while simultaneously permitting native disulfides, which are kinetically favored, to remain substantially intact. The conditions of stage B ultimately allow for both a faster rate of and more complete refolding, by allowing limited formation of mixed native (i.e., correct) and non-native (i.e., incorrect) disulfide bonds, while preventing the formation of such disulfide bonds (in particular, non-native disulfide bonds) from proceeding to a point where they become kinetically trapped, unable to disassociate to free thiols which can reform to native disulfides.

In a particular embodiment of the invention, stage B comprises decreasing the concentration of the denaturant, at pH greater than about 9.5, adding to the solution a disulfide oxidizing compound to a molar concentration sufficient to create mild oxidizing redox conditions to oxidize a portion of the free thiols and form mixed native and non-native disulfide bonds, and adding a human serum albumin refolding ligand at a time from stage (b) to (c). In another embodiment, stage B comprises adjusting the concentration of the denaturant, adding to the solution a disulfide oxidizing compound to a molar concentration equal to or greater than the molar concentration of the total sulfhydryls (protein plus initial reducing agent), at pH greater than about 10, to create mild oxidizing redox conditions and oxidize a portion of the free thiols to form mixed native and non-native disulfide bonds, and adding a human serum albumin refolding ligand at a time from stage (b) to (c). In yet another embodiment, stage B comprises decreasing the concentration of the denaturant and adding to the solution cystine to a molar concentration equal to or greater than the molar concentration of the total sulfhydryls (protein plus initial reducing agent), at a pH of from about 9.5-10.5, to create mild oxidizing redox conditions and oxidize a portion of the free thiols to form mixed native and non-native disulfide bonds, and adding a human serum albumin refolding ligand at a time from stage (b) to (c). In yet another embodiment, stage B comprises decreasing the denaturant concentration, and adding an oxidizing compound to a molar concentration of from about 1.5 to 5 times the molar concentration of the total sulfhydryls (protein plus initial reducing agent), at about pH 9.5 to 10.5, sufficient to create mild oxidizing redox conditions to oxidize a portion of the free thiols and form mixed native and non-native disulfide bonds, and adding an human serum albumin refolding ligand at a time from stage (b) to (c). In yet another embodiment, stage B comprises decreasing the denaturant (such as urea) to a concentration of from about 3 M to 4 M, adding an oxidizing compound to a molar concentration of from about a 2 to 4 times the molar concentration of the total sulfhydryls (protein plus initial reducing agent), at about pH 9.5 to 10.5, sufficient to create mild oxidizing redox conditions to oxidize a portion of the free thiols and form mixed native and non-native disulfide bonds, and adding an human serum albumin refolding ligand at a time from stage (b) to (c).

In stage B of the process, the concentration of the denaturant is decreased and sufficient disulfide oxidizing compound is added to promote partial formation of disulfide bonds, including mixed native and non-native disulfide bonds. The concentration of the denaturant can be decreased by means of dialysis, diafiltration, desalting, or dilution. In the particular embodiments of the present invention, the concentration of the denaturant in the solution of stage A is decreased by diluting with additional amounts of a buffer solution, which may be the same as or compatible with the buffer solution used in stage A. The selection of compatible buffer solutions is routine to those skilled in the art. In the presently disclosed embodiments of the invention, sufficient quantity of additional buffer solution is added to the final solution from stage A to both buffer the solution to a pH of 9.0 to 10.5. In particular embodiments of the invention, the final concentration of the buffer is low ionic strength, from about 5 mM to about 500 mM. In other embodiments, the final concentration is from about 5 mM to about 100 mM. In yet other embodiments of the invention, the final buffer concentration is from about 10 mM to about 50 mM. In yet other embodiments, the final buffer concentration is about 15 mM, which is sufficient to both buffer at this stage and to allow subsequent pH lowering through dilution at step C. Further, low ionic strength at step B promotes optimal refolding at stage C.

In the presently disclosed embodiments of the invention, the buffer solution used to dilute the denaturant also contains a quantity of the same or compatible denaturant, such that the final concentration of the denaturant in the stage B solution is less than the final concentration of the denaturant in the stage A solution. In one embodiment of the present invention, the final concentration of the denaturant in the stage B solution is from about 2 M to about 6 M. In another embodiment, the concentration of the denaturant is from about 2 M to about 4 M. In a particular embodiment, the final concentration of the denaturant in the stage B solution is about 3 M.

In the presently disclosed embodiments, the buffer solution used to dilute the denaturant at stage B also contains a sufficient quantity of an oxidizing compound to create mild oxidizing redox conditions that oxidize a portion of the free thiols to form mixed native and non-native disulfide bonds. Mild oxidizing redox conditions are defined as conditions under which the redox potential is more positive (oxidizing), wherein the equilibrium kinetically favors the formation of disulfide bonds, preferentially retaining substantially all native disulfide bonds and exchanging non-native disulfide bonds, and retaining some free thiols in the HSA protein. The redox potential of the solution (i.e., the extent to which the equilibrium favors oxidization of the free thiols to disulfides) is controlled by the concentration and oxidizing strength of the oxidizing agent relative to the concentration and reducing strength of the free sulfhydryls of the HSA protein.

The oxidizing compound used in stage B of the present invention can be selected from various compounds known to effectively oxidize thiol groups to disulfides. In the presently disclosed embodiments of the invention, the oxidizing compound is a thiol disulfide compound. In particular embodiments of the invention, the thiol disulfide can be cystine, cystamine, oxidized glutathione, oxidized 2-mercaptoethanol, and so forth. In a particular embodiment, the thiol disulfide compound is cystine. A sufficient amount of the thiol disulfide compound is added to create mild oxidizing redox conditions and thereby oxidize a portion of the free thiols to form mixed native and non-native disulfide bonds. In the various embodiments of the present invention, mild oxidizing redox conditions may be created with a molar excess of oxidant relative to total sulfhydryls (protein plus initial reducing agent). Mild oxidizing conditions are achieved by adding a sufficient amount of oxidizing compound that the molar ratio of reductant (such as total cysteine) to oxidant (such as cystine) in stage B is from about 1:1 to about 1:20. In particular, the molar ratio of reductant:oxidant may be from about 1:1 to about 1:12. More particularly, the molar ratio of reductant:oxidant may be from about 1:1 to about 1:5. In a more particular embodiments, the molar ratio of reductant:oxidant is from about 1:3 to about 1:5. For example, at stage B, a 2.5 mg/ml solution of HSA solubilized in 3 M urea and 2 mM dithiothreitol can be brought to mild oxidizing conditions by adding a thiol disulfide oxidizing compound, such as cystine, to a concentration of about 6 mM. As will be apparent to one skilled in the art, the particular concentration of disulfide oxidizing compound will vary according to the particular concentration of total sulfhydryls (protein plus initial reducing agent), denaturant, reducing compound, pH, etc. in order to achieve the desired molar ratio of reductant:oxidant. Generally, under the conditions described in the foregoing example, the concentration of oxidizing compound will be from about 1 mM to about 10 mM. In another embodiment, the concentration of the oxidizing compound is from about 2 mM to about 8 mM. More optimally, the concentration of the oxidizing compound is about 6 mM.

In the embodiments of the present invention disclosed herein, stage B is performed under conditions of high pH, as described above. As used herein, the term “high pH” means a pH at which HSA proteins are capable of forming some secondary structures, including limited formation of mixed native and non-native disulfide bonds, while allowing more efficient refolding at stage C when the pH of the refolding solution is lowered. High pH conditions suitable for stage B are essentially the same as that described above for stage A when a thiol such as DTT is used as the reductant. In the embodiments of the present invention, the pH is greater than about pH 9.0, alternatively greater than about 9.5, and more preferably greater than about 10. In more particular embodiments, the pH is from about 9.5 to about 10.5. In yet other embodiments, the pH is from about 10 to about 10.5.

In a particular embodiment of the invention, the reduced HSA resulting from step A of the process is mixed with buffer solution used in stage A, containing sufficient urea and thiol disulfide compound such that the final concentration of urea is about 3 M, the final concentration of reducing compound from stage A is about 2 mM or less, and the final concentration of thiol disulfide is at least about 6 mM (a three fold molar excess of the diluted DTT at this step).

In another particular embodiment, illustrative of specific conditions, 1 volume of the buffer solution used in stage A above (15 mM sodium bicarbonate, pH 10.3, 6 M urea, 10 mg/ml HSA (i.e., 2.43 mM protein disulfides), 7.3 mM DTT) may be mixed with 3.46 volumes of a buffer solution containing 2.16 M urea and 6.8 mM cystine, to yield a step B buffer solution containing 2.16 mg/ml HSA, 3 M urea, 1.57 mM DTT, and 5 mM cystine. The resulting solution can then be held at 25-37° C. for 1-5 minutes.

Optionally, stage B can be eliminated, but including it allows for both a faster rate of refolding and more complete refolding, theoretically due to preventing kinetically trapped intermediates in the folding pathway in stage C.

While the order of the steps within stage B are illustrated in specific embodiments described above and in the examples set forth below, it is to be understood that such steps may be performed in any particular order without significantly altering the results.

Step C: Refolding

In step C of the process of the present invention, the concentration of the denaturant is further decreased, the pH of the solution is lowered, and a sufficient amount of a second thiol reducing compound is added to the solution, sufficient to create mild reducing conditions and thereby catalyze interchange of the non-native disulfide bonds to native disulfide bonds. The above process provides a human serum albumin protein having substantially native conformation.

In one embodiment of the present invention, stage C comprises further decreasing the concentration of the denaturant, lowering the pH of the solution to less than about 9.5, and adding a second thiol reducing compound to a molar concentration sufficient to create mild reducing conditions to catalyze interchange of the non-native disulfide bonds and form native disulfide bonds, thereby providing a human serum albumin protein having substantially native conformation. In another embodiment, stage C comprises further lowering the concentration of the denaturant, lowering the pH of the solution to less than about 9.5, and adding a second thiol reducing compound to a molar concentration equal to or greater than the molar concentration of the disulfide oxidizing compound, to create mild reducing conditions to catalyze interchange of the non-native disulfide bonds to native disulfide bonds, thereby providing a human serum albumin protein having substantially native conformation. In another embodiment, stage C comprises further decreasing the concentration of the denaturant, lowering the pH of the solution to from about 8.0-9.5, adding an HSA refolding ligand, and adding to the solution cysteine to a molar concentration equal to or greater than the molar concentration of the disulfide oxidizing compound, to create mild reducing conditions, and catalyze interchange of the non-native disulfide bonds to native disulfide bonds, thereby providing a human serum albumin protein having substantially native conformation. In another embodiment, stage C comprises further decreasing the urea concentration, lowering the pH to about 8.5-9.0, and adding cysteine to a molar concentration equal to or greater than the molar concentration of cystine, sufficient to create mild reducing conditions to catalyze interchange of the non-native disulfide bonds to native disulfide bonds, thereby providing a human serum albumin protein having substantially native conformation. In another embodiment, stage C comprises further decreasing the urea concentration to less than about 1.5 M, lowering the pH to from about 8.5 to 9.0, and adding cysteine to a molar concentration of equal to or greater than about 2 times the molar concentration of cystine, sufficient to create mild reducing conditions and catalyze interchange of the non-native disulfide bonds to native disulfide bonds, thereby providing a human serum albumin protein having substantially native conformation.

The concentration of the denaturant is further reduced by means of dialysis, diafiltration, desalting, or dilution. In the presently disclosed embodiments of the present invention, the concentration of the denaturant in the solution from step B is decreased by diluting with additional buffer solution, which may be the same as or compatible with the buffer solution used in prior steps. The selection of compatible buffer solutions is routine to those skilled in the art. In one embodiment of the invention, the buffer is compatible with any buffer with a pKa near pH 9.0. Exemplary buffers include sodium bicarbonate, sodium borate, sodium phosphate, Tris, and ammonium acetate. In the presently disclosed embodiments of the invention, a sufficient quantity of additional buffer solution is added to the final solution from step B to both buffer the solution and reduce the pH to a level at which complete formation of native disulfide bonds can occur. In a particular embodiment, effective buffer concentrations will be 150 mM or less. In particular embodiments of the invention, the buffer is adjusted to a concentration of from about 15 to about 500 mM. In other embodiments, the buffer concentration is from about 50 mM to about 350 mM. In yet other embodiments of the invention, the buffer concentration is from about 100 mM to about 250 mM. In yet other embodiments, the buffer concentration is about 150 mM, which is sufficient to both buffer at this step and to allow subsequent pH lowering through dilution at step C.

In step C of the process, the pH of the solution is lowered. In the presently disclosed embodiments, the pH of the solution is lowered to a level still considered a “high pH.” In these embodiments, the pH is less than 10, and may range from about 8.0 to about 10. In more particular embodiments, the pH is from about pH 9.0 to about pH 9.5. Because dilution of bicarbonate results in an increase of the pH, it may be necessary to make corresponding adjustments in the pH of the buffer that is added in step C.

Dilution of the solution from step B by addition of buffer is also done for the purpose of decreasing the urea concentration, which has the effect of further promoting formation of native structure and native disulfide bonds in the HSA protein. In the disclosed embodiments of the invention, sufficient buffer is added to decrease the urea to a concentration less than about 3 M. In another embodiment, sufficient buffer is added to decrease the urea to a concentration to from about 0.3 M to about 3 M. In particular embodiments, the urea concentration of the solution of step C is brought to 0.3 M to about 1.5 M. Without sodium caprate ligand, the refolding rate increases as the urea concentration decreases. With 0.5-25 mM sodium caprate, the refolding rate is essentially the same from 0.3-1.5 M urea.

In step C of the present invention, a second reducing agent is also added. In particular embodiments of the invention, the reducing compound is a thiol reducing compound. The thiol reducing agent can be DTT, cysteine, cysteamine, glutathione, 2-mercaptoethanol, and equivalents. In a particular embodiment, the thiol reducing agent is cysteine. A sufficient amount of the thiol reducing agent is added to create mild reducing redox conditions and thereby catalyze the interchange of the non-native disulfide bonds to native disulfide bonds. Mild reducing redox conditions are defined as conditions under which the redox potential is neutral to negative (reducing), wherein the equilibrium kinetically favors essentially complete formation of native disulfide bonds of the HSA protein. The redox potential of the solution is controlled by the concentration of the first and second reducing compounds relative to the oxidizing compound used in step B. Such conditions are achieved by adding a sufficient amount of reducing compound to bring the molar ratio of reductant (such as cysteine) to oxidant (such as cystine) in Step B to from about 1:1 to about 5:1. In particular, the molar ratio of reductant:oxidant may be from about 1:1 to about 4:1. More particularly, the molar ratio of reductant:oxidant may be from about 1:1 to about 3:1. More particularly, the molar ratio of reductant:oxidant may be from about 1:1 to about 2:1. It has been found that essentially redox neutral conditions (molar ratio of reductant:oxidant essentially 2:1) are suitable for promoting refolding in Step C. In particular embodiments, starting with a solution having a concentration of cysteine reducing compound from about 1.5 to about 15 mM cysteine, the concentration of cystine oxidizing compound may be from about 0.75 to about 10 mM cystine. In more particular embodiments, where the concentration of cysteine reducing compound being from about 3 mM to about 12 mM cysteine and the concentration of cystine oxidizing compound is from about 1.5 mM to about 6 mM cystine. In yet another embodiment, where the concentration of cysteine reducing compound is about 6 mM and the concentration of cystine oxidizing compound is about 3 mM.

With respect to the protein concentration, HSA may be present in the solution of step C in a concentration of from about 0.1 mg/ml to about 50 mg/ml. In other embodiments, the HSA protein concentration can be from about 0.5 mg/ml to about 20 mg/ml. In more particular embodiments, the HSA protein concentration can be from about 2 mg/ml to about 15 mg/ml. In yet another particular embodiment, the HSA protein concentration at step C can be up to about 10 mg/ml.

While the order of the steps within stage C are illustrated in specific embodiments described above and in the examples set forth below, it is to be understood that such steps may be performed in any particular order without significantly altering the results.

Pharmaceutical Preparations of HSA

The HSA thus obtained may be made into pharmaceutical preparations by generally known means such as 10 hours of heat sterilization at 60° C., ultrafiltration, filter sterilization, dispensation, freeze-drying and the like. An illustrative example of the pharmaceutical preparation of the present invention is a liquid preparation which contains HSA in an amount of 5 to 25%, has a pH of approximately 6.4 to 7.4 and has an osmotic pressure ratio of around 1.

The HSA-containing pharmaceutical preparation of the instant invention may contain stabilizers which include acetyltryptophan or a salt thereof (e.g., sodium salt) and sodium caprylate. Each stabilizer may be used in an amount of approximately 0.001 to 0.2 M, preferably 0.01 to 0.05 M in a 25% HSA solution. The sodium content may be 3.7 mg/ml or less. The HSA preparation may further contain pharmaceutically acceptable additives such as sodium chloride and the like.

In general, the stabilizers may be added prior to the aforementioned preparation steps such as 10 hours of heat sterilization at 60° C., ultrafiltration, filter sterilization, dispensation, freeze-drying and the like. Therefore, not only preservation stability of HSA but also its stability during the preparation process of the pharmaceutical preparation of the instant invention can be improved.

The HSA-containing pharmaceutical preparation thus obtained can be used clinically as injections in the same manner as the case of the prior art plasma-derived HSA preparations. For example, it may be used for the purpose of rapidly increasing blood volume, mainly at the time of shock, supplementing circulation blood volume, improving hypoproteinemia or maintaining plasma osmotic pressure. More illustratively, the HSA-containing pharmaceutical preparation of the present invention can be used effectively for the treatment of hypoalbuminemia caused by the loss of albumin (burn injury, nephrotic syndrome or the like) or by the reduction of albumin synthesizing ability (hepatic cirrhosis or the like), as well as for the treatment of hemorrhagic shock and the like.

The pharmaceutical preparation may be administered gradually by intravenous injection or intravenous drip infusion, with a dose of generally from 20 to 50 ml as a 25% HSA solution (5 to 12.5 g as HSA) per one administration for an adult. The dose may be changed optionally depending on the age, symptoms, weight and the like of the patient. Properties of the purified recombinant HSA.

### EXAMPLE 1

### HSA Refolding

A process for refolding HSA is illustrated in the following example:

Step A. Buffer A (sodium bicarbonate 15 mM, pH 10.3) is first prepared. To Buffer A is added urea to a concentration of 6 M, and DTT to a concentration of 7.3 mM. Human serum albumin (HSA) protein is then added to a concentration of 10 mg/ml (2.43 mM protein disulfides), and allowed to reduced 37° C. for 15-30 minutes, resulting in Solution A.

Step B. Buffer B (sodium bicarbonate 15 mM, pH 10.3, 2.16 M urea and 7.7 mM cystine) is prepared. 1 volume of Solution A is then mixed with 3.46 volumes of Buffer B, to yield Solution B (HSA 2.16 mg/ml, urea 3 M, DTT [or equivalents] 1.57 mM, and cystine 6 mM). Solution B is held at 37° C. for 1-5 minutes.

Step C. Continuing with the example from above, 1 volume of Solution B is mixed with 1 volume of Buffer C (100 mM sodium bicarbonate, pH 9.0, containing no urea, and 12 mM cysteine), to yield Solution C (HSA 1.08 mg/ml; 57.5 mM sodium bicarbonate, pH 9.3; urea 1.5 M; cystine 3 mM; cysteine 6 mM), to yield 10-20% refolding of HSA after 30 minutes 37° C.

### EXAMPLE 2

### HSA Refolding with Ligand Assistance

The HSA refolding process of the present invention may also be performed using an HSA refolding ligand to assist the refolding process, as described below.

Step A. Buffer A (sodium bicarbonate 15 mM, pH 10.3) is first prepared. To Buffer A is added urea to a concentration of 6 M, and DTT to a concentration of 7.3 mM. Human serum albumin (HSA) protein is then added to a concentration of 10 mg/ml (2.43 mM protein disulfides), and allowed to reduced 37° C. for 15-30 minutes, resulting in Solution A.

Step B. Buffer B (sodium bicarbonate 15 mM, pH 10.3, 2.16 M urea and 7.7 mM cystine) is prepared. 1 volume of Solution A is then mixed with 3.46 volumes of Buffer B, to yield Solution B (HSA 2.16 mg/ml, urea 3 M, DTT [or equivalents] 1.57 mM, and cystine 6 mM). Sodium caprate is added to Solution B to a final concentration of 10 mM. Solution B is held at 37° C. for 1-5 minutes.

Step C. Continuing with the example from above, 1 volume of Solution B is mixed with 1 volume of Buffer C (100 mM sodium bicarbonate, pH 9.0, containing no urea, and 12 mM cysteine), to yield Solution C (HSA 1.08 mg/ml; 57.5 mM sodium bicarbonate, pH 9.3; urea 1.5 M; cystine 3 mM; cysteine 6 mM). Sodium caprate is added to Solution C to a final concentration of 10 mM. Solution C is held at 37° C. for a period of 30 minutes. HSA protein is refolded to native conformation in yield in excess of 80-90%.

It is to be understood that the foregoing descriptions of embodiments of the present invention are exemplary and explanatory only, are not restrictive of the invention, as claimed, and merely illustrate various embodiments of the invention. It will be appreciated that other particular embodiments consistent with the principles described in the specification but not expressly disclosed may fall within the scope of the claims.

1. A process for refolding and renaturing human serum albumin protein to substantially native conformation, comprising the following stages:
(a) solubilizing human serum albumin protein in a solution comprising a denaturant and a first thiol reducing compound at concentrations sufficient to disrupt formation of all disulfide bonds and form free thiols;
(b) decreasing the concentration of the denaturant, at pH greater than about 9.5, adding to the solution a disulfide oxidizing compound to a molar concentration sufficient to create mild oxidizing redox conditions to oxidize a portion of the free thiols and form mixed native and non-native disulfide bonds, and adding a human serum albumin refolding ligand at a time from stage (b) to (c); and
(c) further decreasing the concentration of the denaturant, lowering the pH of the solution to less than 9.5, and adding a second thiol reducing compound to a molar concentration sufficient to create mild reducing conditions to catalyze interchange of the non-native disulfide bonds and form native disulfide bonds,
thereby providing a human serum albumin protein having substantially native conformation.

2. A process according to claim 1, wherein the HSA refolding ligand binds to native HSA site 2.

3. A process according to claim 2, wherein the HSA refolding ligand is selected from the group consisting of alkyl fatty acids, and salts thereof, having 6 to 12 carbon atoms.

4. A process according to claim 3, wherein the binding ligand is sodium caprate.

5. A process according to claim 4, wherein the sodium caprate concentration is from about 0.5 to about 25 mM.

6. A process according to claim 5, wherein the binding ligand is selected from the group consisting of alkyl alcohols having from 6 to 12 carbon atoms.

7. A process for refolding and renaturing human serum albumin protein to substantially native conformation, comprising the following stages:
(a) solubilizing human serum albumin protein in a solution comprising a denaturant, and a first thiol reducing compound at a molar concentration greater than the molar concentration of human serum albumin disulfide bonds, sufficient to disrupt formation of all disulfide bonds and form free thiols;
(b) decreasing the concentration of the denaturant, adding to the solution a disulfide oxidizing compound to a molar concentration equal to or greater than the molar concentration of the total sulfhydryls (protein plus initial reducing agent), at pH greater than about 9.5, to create mild oxidizing redox conditions and oxidize a portion of the free thiols to form mixed native and non-native disulfide bonds, and adding a human serum albumin refolding ligand at a time from stage (b) to (c); and
(c) further decreasing the concentration of the denaturant, lowering the pH of the solution to less than about 9.5, and adding a second thiol reducing compound to a molar concentration equal to or greater than the molar concentration of the disulfide oxidizing compound, to create mild reducing conditions to catalyze interchange of the non-native disulfide bonds to native disulfide bonds,
thereby providing a human serum albumin protein having substantially native conformation.

8. A process according to claim 7, wherein the HSA refolding ligand binds to native HSA site 2.

9. A process according to claim 8, wherein the HSA refolding ligand is selected from the group consisting of alkyl fatty acids, and salts thereof, having 6 to 12 carbon atoms.

10. A process according to claim 9, wherein the binding ligand is sodium caprate.

11. A process according to claim 10, wherein the sodium caprate concentration is from about 0.5 to about 25 mM.

12. A process according to claim 8, wherein the binding ligand is selected from the group consisting of alkyl alcohols having from 6 to 12 carbon atoms.

13. A process for refolding and renaturing human serum albumin protein to substantially native conformation, comprising the following stages:
(a) solubilizing human serum albumin protein in a solution comprising a denaturant, and a first thiol reducing compound at a molar concentration greater than the molar concentration of human serum albumin disulfide bonds, sufficient to disrupt formation of all disulfide bonds and form free thiols;
(b) decreasing the concentration of the denaturant and adding to the solution cystine to a molar concentration equal to or greater than the molar concentration of the total sulfhydryls (protein plus initial reducing agent), at a pH of from about 9.5-10.5, to create mild oxidizing redox conditions and oxidize a portion of the free thiols to form mixed native and non-native disulfide bonds, and adding a human serum albumin refolding ligand at a time from stage (b) to (c); and
(c) further decreasing the concentration of the denaturant, lowering the pH of the solution to from about 8.0-9.5, adding an HSA refolding ligand, and adding to the solution cysteine to a molar concentration equal to or greater than the molar concentration of the disulfide oxidizing compound, to create mild reducing conditions, and catalyze interchange of the non-native disulfide bonds to native disulfide bonds,
thereby providing a human serum albumin protein having substantially native conformation.

14. A process according to claim 13, wherein the HSA refolding ligand binds to native HSA site 2.

15. A process according to claim 14, wherein the HSA refolding ligand is selected from the group consisting of alkyl fatty acids, and salts thereof, having 6 to 12 carbon atoms.

16. A process according to claim 15, wherein the binding ligand is sodium caprate.

17. A process according to claim 16, wherein the sodium caprate concentration is from about 0.5 to about 25 mM.

18. A process according to claim 14, wherein the binding ligand is selected from the group consisting of alkyl alcohols having from 6 to 12 carbon atoms.

19. A process for refolding and renaturing human serum albumin protein to substantially native conformation, comprising the following stages:
(a) solubilizing human serum albumin protein in a solution comprising a denaturant, and a first thiol reducing compound to a molar concentration greater than the molar concentration of human serum albumin disulfide bonds, sufficient to disrupt formation of all disulfide bonds and form free thiols;
(b) decreasing the denaturant concentration, and adding cystine to a molar concentration of from about 1.5 to 5 times the molar concentration of the total sulfhydryls (protein plus initial reducing agent), at about pH 9.5 to 10.5, sufficient to create mild oxidizing redox conditions to oxidize a portion of the free thiols and form mixed native and non-native disulfide bonds, and adding a human serum albumin refolding ligand at a time from stage (b) to (c); and
(c) further decreasing the urea concentration, lowering the pH to about 8.5-9.5, and adding cysteine to a molar concentration equal to or greater than the molar concentration of cystine, sufficient to create mild reducing conditions to catalyze interchange of the non-native disulfide bonds to native disulfide bonds,
thereby providing a human serum albumin protein having substantially native conformation.

20. A process according to claim 19, wherein the HSA refolding ligand binds to native HSA site 2.

21. A process according to claim 20, wherein the HSA refolding ligand is selected from the group consisting of alkyl fatty acids, and salts thereof, having 6 to 12 carbon atoms.

22. A process according to claim 21, wherein the binding ligand is sodium caprate.

23. A process according to claim 22, wherein the sodium caprate concentration is from about 0.5 to about 25 mM.

24. A process according to claim 20, wherein the binding ligand is selected from the group consisting of alkyl alcohols having from 6 to 12 carbon atoms.

25. A process for refolding and renaturing human serum albumin protein to substantially native conformation, comprising the following stages:
(a) solubilizing human serum albumin protein in a solution comprising urea, and a first thiol reducing compound to a molar concentration greater than the molar concentration of human serum albumin disulfide bonds, sufficient to disrupt formation of all disulfide bonds and form free thiols;
(b) decreasing the urea to a concentration of from about 3 M to 4 M, adding a disulfide oxidizing compound such as cystine to a molar concentration of from about a 2 to 4 times the molar concentration of the total sulfhydryls (protein plus initial reducing agent), at about pH 9.5 to 10.5, sufficient to create mild oxidizing redox conditions to oxidize a portion of the free thiols and form mixed native and non-native disulfide bonds, and adding a human serum albumin refolding ligand at a time from stage (b) to (c); and
(c) further decreasing the urea concentration to less than about 1.5 M, lowering the pH to from about 8.5 to 9.5, and adding cysteine to a molar concentration of equal to or greater than about 2 times the molar concentration of cystine, sufficient to create mild reducing conditions and catalyze interchange of the non-native disulfide bonds to native disulfide bonds,
thereby providing a human serum albumin protein having substantially native conformation.

26. A process according to claim 25, wherein the HSA refolding ligand binds to native HSA site 2.

27. A process according to claim 26, wherein the HSA refolding ligand is selected from the group consisting of alkyl fatty acids, and salts thereof, having 6 to 12 carbon atoms.

28. A process according to claim 27, wherein the binding ligand is sodium caprate.

29. A process according to claim 28, wherein the sodium caprate concentration is from about 0.5 to about 25 mM.

30. A process according to claim 26, wherein the binding ligand is selected from the group consisting of alkyl alcohols having from 6 to 12 carbon atoms.

31. A process for refolding and renaturing human serum albumin protein to substantially native conformation, comprising combining denatured human serum albumin and a human serum albumin site 2 binding ligand in a solution comprising a denaturant, a first thiol reducing compound, and a disulfide oxidizing compound at molar concentrations sufficient to create mild reducing conditions to catalyze interchange of the non-native disulfide bonds and form native disulfide bonds.

32. A process according to claim 31, wherein the binding ligand is selected from the group consisting of salts of alkyl fatty acids, and salts thereof having from 6 to 12 carbon atoms.

33. A process according to claim 32, wherein the binding ligand is sodium caprate.

34. A process according to claim 33, wherein the sodium caprate concentration is from about 0.5 to about 25 mM.

35. A process according to claim 34, wherein the binding ligand is selected from the group consisting of alkyl alcohols having from 6 to 12 carbon atoms.

36. A process according to claim 31, wherein the site 2 ligand is selected from the group consisting of Ibuprofen and Naproxen.

37. A process for refolding and renaturing human serum albumin protein to substantially native conformation, comprising combining denatured human serum albumin and a human serum albumin site 2 binding ligand in a solution under conditions conducive to refolding of human serum albumin.

38. A process according to claim 37, wherein the binding ligand is selected from the group consisting of salts of alkyl fatty acids, and salts thereof having from 6 to 12 carbon atoms.

39. A process according to claim 38, wherein the binding ligand is sodium caprate.

40. A process according to claim 39, wherein the sodium caprate concentration is from about 0.5 to about 25 mM.

41. A process according to claim 37, wherein the binding ligand is selected from the group consisting of alkyl alcohols having from 6 to 12 carbon atoms.

42. A process according to claim 41, wherein the site 2 ligand is selected from the group consisting of Ibuprofen and Naproxen.
